(19) United States
(12) Patent Application Publication
(10) Pub. No.: US 2022/0281836 A1
(43) Pub. Date: Sep. 8, 2022

(54) PROCESS FOR THE PREPARATION OF APALUTAMIDE

(71) Applicant: DR. REDDY'S LABORATORIES LIMITED, Hyderabad (IN)

(72) Inventors: Abhishek SUD, Hyderabad (IN); Nm SEKHAR, Hyderabad (IN); Rajeev REHANI, Hyderabad (IN); Babu IRENI, Hyderabad (IN); Sateesh MADAVARAM, Nagarkurnool (IN); Narsihma Reddy CHADA, Gajwel (IN); Ashok ARIGE, Hyderabad (IN)

(21) Appl. No.: 17/636,634

(22) PCT Filed: Aug. 14, 2020
(86) PCT No.: PCT/IB2020/057659

§ 371 Date: Feb. 18, 2022

(30) Foreign Application Priority Data
Aug. 22, 2019 (IN) 201941033825
Jan. 30, 2020 (IN) 202041004075

(51) Int. Cl. C07D 401/04

(52) U.S. Cl. CPC C07D 401/04; C07B 2200/13

(57) ABSTRACT

Aspect of the present application relates to process for the preparation of crystalline form of Apalutamide and process for the preparation of Apalutamide in the presence of neutralizing agent selected from triethylsilylchloride, trimethylsilyl chloride, zinc chloride, aluminium chloride, iron chloride, sodium chloride, acetic acid, ammonium chloride or mixture thereof followed by treating with acid to obtain Apalutamide.

### FIELD OF THE INVENTION

Aspect of the present application relates to process for the preparation of crystalline form of Apalutamide and process for the preparation of Apalutamide in the presence of neutralizing agent.

### BACKGROUND OF THE INVENTION AND DISCLOSURE OF PRIOR ART

The drug compound having the adopted name “Apalutamide” has chemical name: 4-(7-(6-cyano-5-(trifluorom ethyl)pyrid in-3-yl)-8-oxo-6-th ioxo-5, 7-d iazaspiro [3.4]octan-5-yl)-2-fluoro-N-methylbenza-mide, has the following chemical structure:

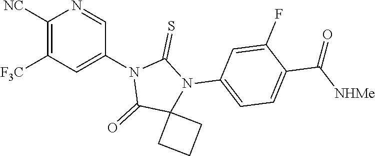 [SMILES: CNC(=O)c1ccc(N2C(=S)N(c3cnc(C#N)c(C(F)(F)F)c3)C(=O)C23CCC3)cc1F]

Apalutamide is approved in US as ERLEADA tablet for oral administration for the treatment of patients with non-metastatic castration-resistant prostate cancer (NM-CRPC). ERLEADA is available as 60 mg tablet and recommended daily dose of 240 mg.

U.S. Pat. No. 8,445,507B2 discloses apalutamide, method for treating prostate cancer using apalutamide and its pharmaceutical composition.

U.S. Pat. No. 8,445,507B2 discloses process for the preparation of apalutamide by reacting 5-isothiocyanato-3-(trifluoromethyl)picolinonitrile with 4-(1-cyanocyclobutylamino)-2-fluoro-N-methylbenzamide in microwave. The synthetic approach is very limited for industrial application because microwave is not easy to apply in large scale synthesis and results in higher costs. The synthetic approach is described below.

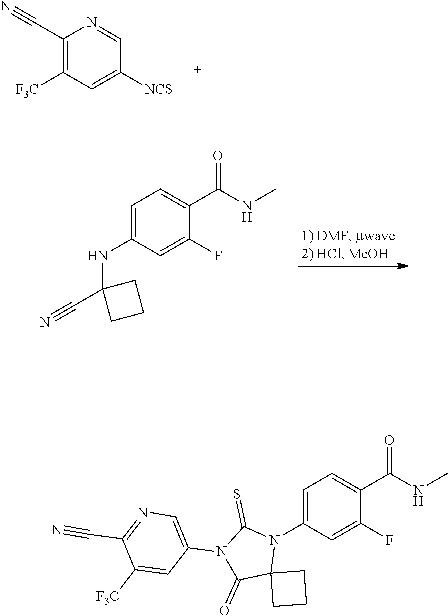

U.S. Pat. No. 9,481,663B2 discloses crystalline Form B of apalutamide and process for the preparation of crystalline Form B of apalutamide using water, ethyl acetate, tert-butyl methyl ether (TBME), toluene, isopropylacetate, or methyl ethyl ketone (MEK) as solvents.

WO2013184681A1 discloses crystalline Form A, Form B, Form C, Form D, Form E, Form F, Form G, Form H, Form I and Form J of apalutamide.

WO2016124149A1 discloses crystalline Form I and Form II of apalutamide.

WO2018112001A1 discloses crystalline Form T2, Form T6, Form T11 and Form T13 of apalutamide.

WO2019135254A1 discloses crystalline Form M4, Form M5, Form M6 of apalutam ide.

Comprehensive systematic polymorph screening in drug development and the selection of the most suitable crystal form are one of the important research contents that cannot be ignored. Identifying more cost effective and industrially viable process of stable crystalline form of apalutamide also cannot be ignored. Although approaches for preparing apalutamide have been disclosed as discussed above, there is still an unmet need for a more environment friendly, industrially practical, and economical process for preparation of apalutamide. The present process disclosed herein address this need and other needs.

### SUMMARY OF THE INVENTION

In one embodiment, the present invention provides a process for the preparation of apalutamide, the process comprising reacting formula III with formula IV in the presence of neutralizing agent followed by treating with acid to obtain Apalutamide. The synthetic approach is described below.

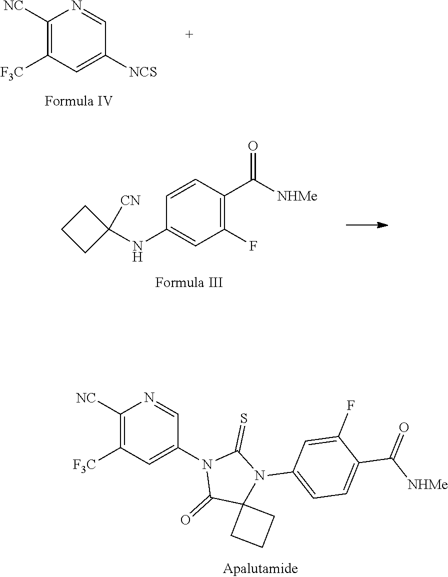

In second embodiment, the present invention provides a process for the preparation of crystalline form of apalutamide characterized by a PXRD pattern comprising peaks at about 12.1°, 16.0°, 16.7°, 20.1°20.3°±0.1°2θ, comprising the steps of:

a) providing apalutamide in solvent selected from n-butanol, methanol, diisopropyl ether, isobutyl acetate, n-pentanol, methyl tert-butyl ether or mixture thereof; and

b) isolating crystalline form of apalutamide.

### BRIEF DESCRIPTION OF THE DRAWING

FIG. 1 is an illustrative X-ray powder diffraction pattern of amorphous form of apalutamide prepared by the method of example No 4.

FIG. 2 is an illustrative X-ray powder diffraction pattern of crystalline form of apalutamide prepared by the method of example No 5.

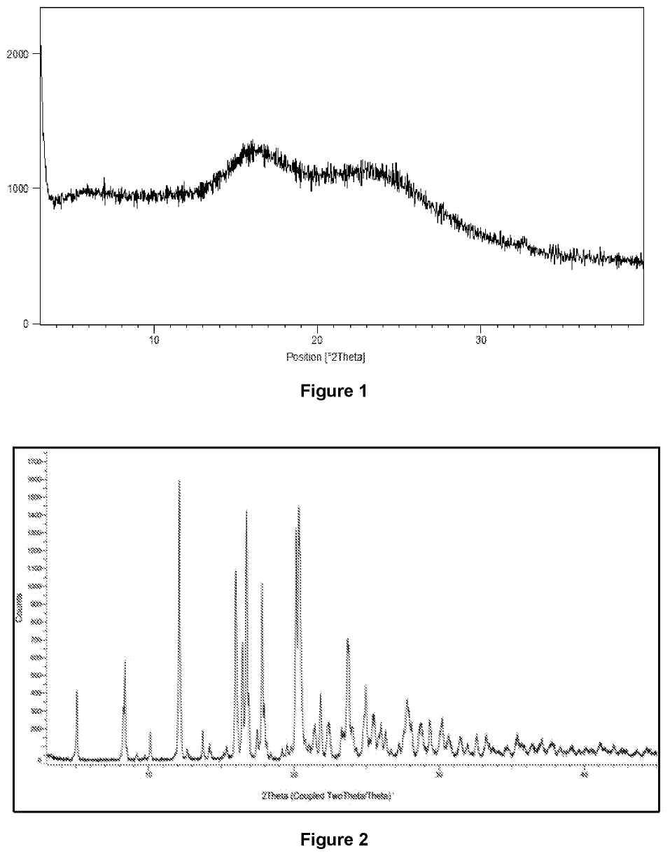

### DETAILED DESCRIPTION OF THE INVENTION

In one embodiment, the present invention provides a process for the preparation of apalutamide, the process comprising reacting formula III with formula IV in the presence of neutralizing agent followed by treating with acid to obtain Apalutamide. The synthetic approach is described below.

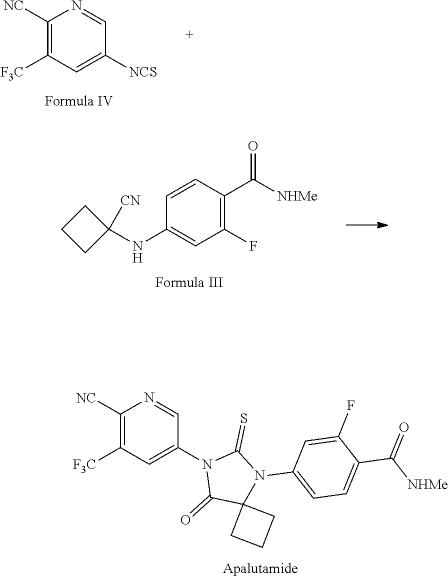

In an aspect of the present invention, the condensation process can be carried out in the presence of any suitable neutralizing agent including but not limited to: triethylsilyl chloride, trimethylsilyl chloride zinc chloride, aluminium chloride, iron chloride, borontriflouride etherate (BF3.OEt2), titanium isopropoxide, sodium chloride, acetic acid or ammonium chloride or mixture thereof.

In an aspect of the present invention, the condensation process can be carried out in any suitable solvent including but not limited to: toluene, N,N-dimethyl acetamide(DMA), acetonitrile, ethyl acetate, dimethylformam ide(DMF), dimethyl sulfoxide(DMSO), 2-methyl tetrahydrofuran, Isopropyl acetate, tetrahydrofuran(THF), chlorobenzene or mixture thereof.

In an aspect of the present invention, the condensation process can be carried out in the presence of any suitable acids including but not limited to: hydrochloric acid (HCl), hydrofluoric acid (HF), hydrobromic acid (HBr), hydroiodic acid (HI),sulfuric acid (H2SO4), nitric acid (HNO3), phosphoric acid (H3PO4) or mixture thereof.

In an aspect of the present invention, the condensation process can be carried out at a temperature ranging from about 0° C. to about 120° C. Preferably the condensation reaction is carried out at a temperature ranging from about 20° C. to about 70° C. The time required for the reaction may also vary widely, depending on many factors, notably the reaction temperature and the nature of the reagents and solvent employed. However, provided that the reaction is effected under the conditions out lined above, a period of from about 10 minutes to about 48 hours or longer.

During the condensation of Formula III with Formula IV, imine anion is generated as an intermediate. This anion reacts with the 2^{nd} molecule of Formula IV to stabilize. This has the disadvantage of using up to 3 equivalents of Formula IV as part of the process. During the process development it was unexpectedly discovered that neutralizing agent is useful for reducing the equivalents of Formula IV from 3 to 1.7

A specific process for the preparation of apalutamide by a method of present application can be illustrated as given in below Schemes 1, 2 and 3.

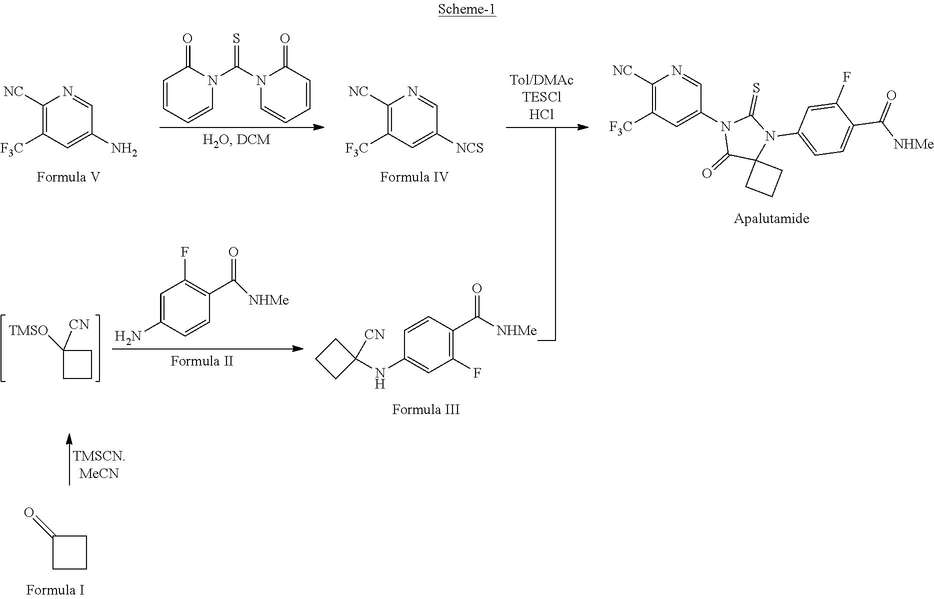

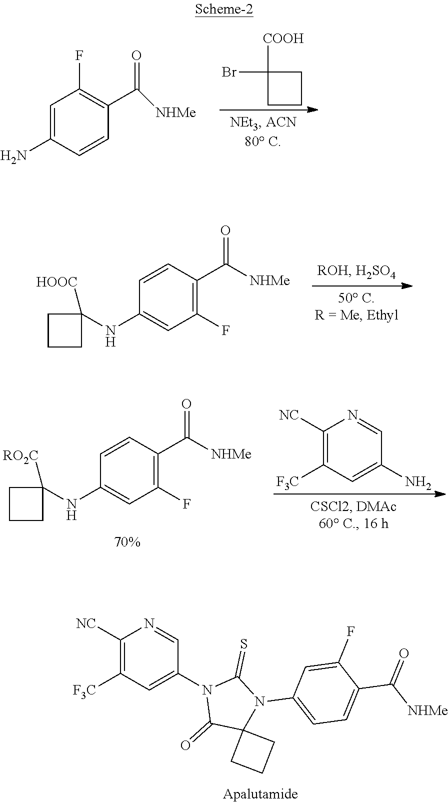

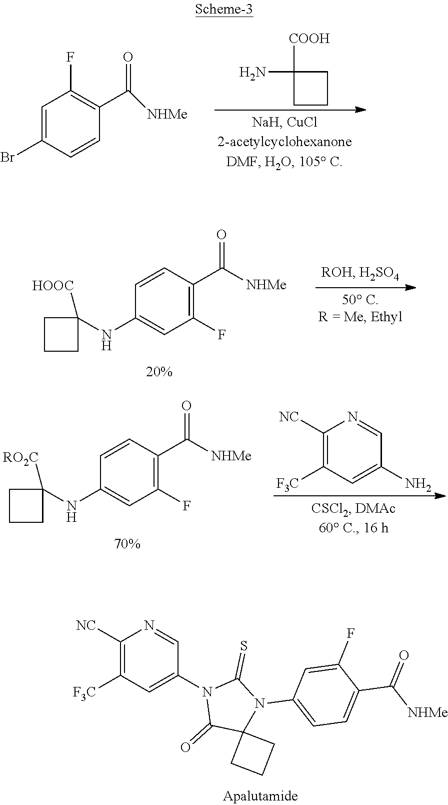

The starting materials (Formula II, III and IV) of apalutamide can be prepared by any known method or by the process that is illustrated as given below in schemes:

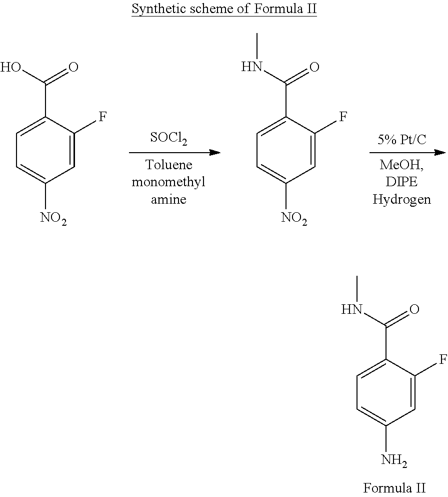

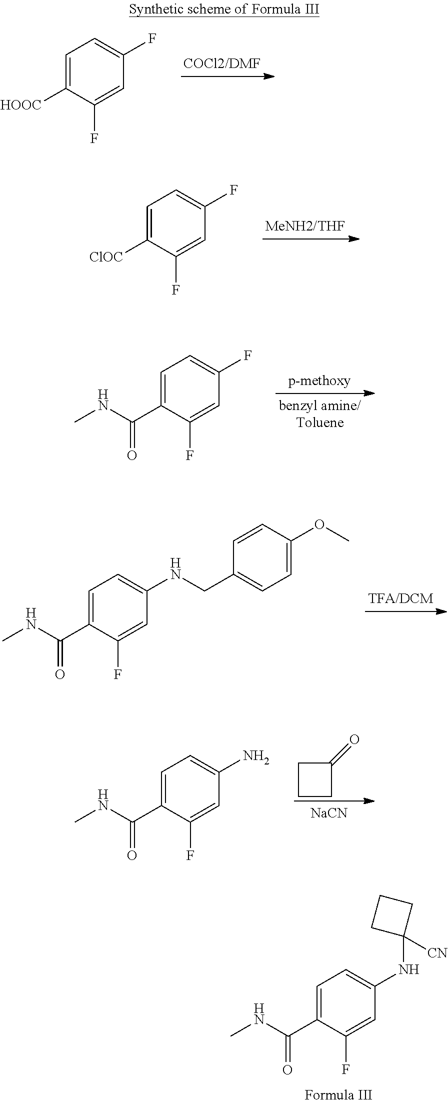

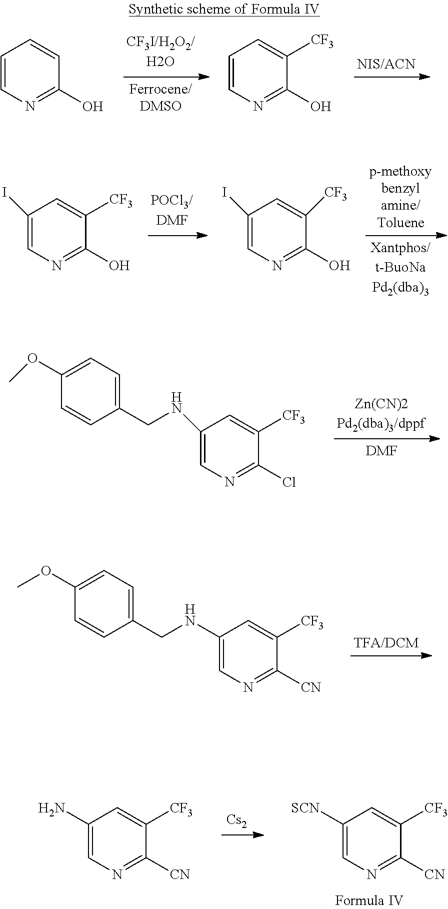

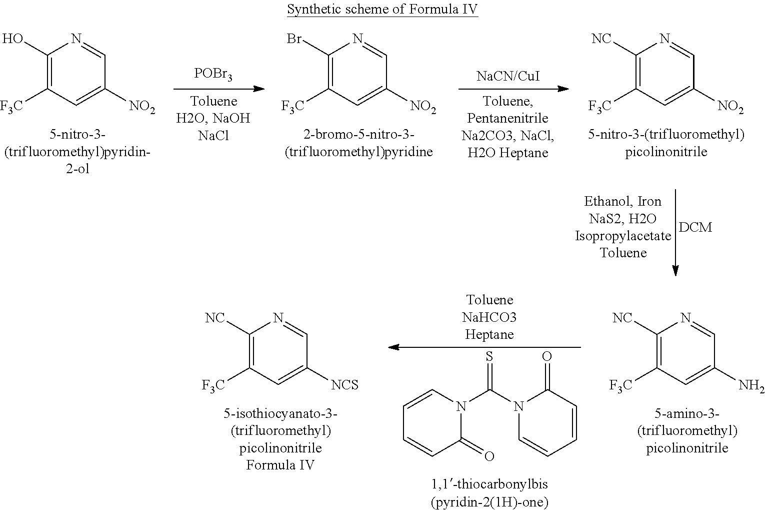

In an aspect, apalutamide may be prepared with or without isolation of intermediates.

In second embodiment, the present invention provides process for the preparation of crystalline form of apalutamide characterized by a PXRD pattern comprising peaks at about 12.1°, 16.0°, 16.7°, 20.1°20.3°±0.1 °2θ, comprising the steps of:

a) providing apalutamide in solvent selected from n-butanol, methanol, diisopropyl ether, isobutyl acetate, n-pentanol, methyl tert-butyl ether or mixture thereof; and

b) isolating crystalline form of apalutamide.

In an aspect of the present invention, step a) may be carried out by dissolving apalutamide in solvent or by taking the reaction mixture containing apalutamide directly.

In an aspect of the present invention, a solution of apalutamide can be prepared at any suitable temperatures, such as about 10° C. to about the reflux temperature of the solvent used. Stirring and heating may be used to reduce the time required for the dissolution process.

In an aspect of the present invention, a solution of apalutamide may be filtered to make it clear, free of unwanted particles.

In an aspect of the present invention, the obtained solution may be optionally treated with an adsorbent material, such as carbon and/or hydrose, to remove colored components, etc., before filtration.

In an aspect of the present invention, the seed crystals of apalutamide is optionally added to the mixture of apalutamide and suitable solvent. When the seed crystals are added, they are added in a quantity from about 0.1% w/w to about 50% w/w over the weight of free base. Specifically, the seed crystals are added in a quantity from about 0.5% to about 20% w/w and more specifically the seed crystals are added in a quantity from about 1% to about 10% w/w.

In an aspect of the present invention, the solution of apalutamide may be cooled to a suitable temperature before and/or after contacting with seed crystals.

In an aspect of the present invention, a solution of apalutamide may be optionally contacted with an anti-solvent. Anti-solvent may include, but not limited to n-hexane, n-heptane, cyclohexane, water or mixtures thereof.

In an aspect of the present invention, the anti-solvent may be contacted at suitable temperature for the nucleation of solids and for sufficient time for the formation of solids. The anti-solvent may be contacted in sufficient quantity to complete the formation of solids.

In an aspect of the present invention, the solution of aplutamide may be cooled to a suitable temperature before and/or after contacting with anti-solvent.

In an aspect of the present invention, isolation of crystalline form of apalutamide may be carried out by any methods known in the art or procedures described in the present application. In an aspect of the present invention, crystalline Form of apalutamide may be isolated by employing any of the techniques, but not limited to: decantation, filtration by gravity or suction, centrifugation, adding solvent to make slurry followed by filtration, or other techniques specific to the equipment used and the like, and optionally washing with a solvent.

In an aspect of the present invention, drying crystalline Form of apalutamide may be carried out at temperatures and times sufficient to achieve desired quality of product. Drying may be carried out for any time period required for obtaining a desired quality, such as from about 5 minutes to 10 hours or longer.

Starting materials used for the preparation of crystalline form of apalutamide may be any crystalline or amorphous in nature. Further, these starting material may be purified according to any of the method known in the art such as recrystallization, slurrying, acid-base treatment i.e., salt making and breaking, chromatography, fractional distillation or any other separation methods, before using. Apalutamide that may be used as the input for the process of the present invention may be obtained by the processes described in the art. For example apalutamide may be prepared by the processes described in U.S. Pat. Nos. 8,445,507B2, 8,987,452B2 or IN201941033825.

In another aspect, the present application provides crystalline form of apalutamide having chemical purity may be more than 99% by HPLC or more than 99.5% by HPLC or more than 99.9% by HPLC.

In another aspect, the present application provides crystalline form of apalutamide having particle size (D90) may be less than 100 microns or less than 50 microns or less than 20 microns.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application. Potential Impurities possible in apalutamide are described in the present application and can have structures as illustrated below.

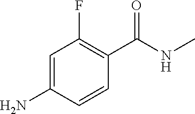

4-Amino-2-fluoro-N-methylbenzamide

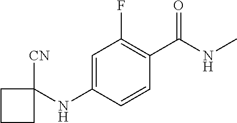 [SMILES: CNC(=O)c1ccc(NC2(C#N)CCC2)cc1F]

4-((1-cyanocyclobutyl)amino)-2-fluoro-N-methylbenzamide

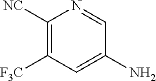 [SMILES: N#Cc1ncc(N)cc1C(F)(F)F]

5-amino-3-(trifluoromethyl)picolinonitrile

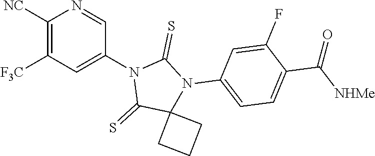 [SMILES: CNC(=O)c1ccc(N2C(=S)N(c3cnc(C#N)c(C(F)(F)F)c3)C(=S)C23CCC3)cc1F]

4-(7-(6-cyano-5-(trifluoromethyl)pyridin-3-yl)-6, 8-dithioxo-5, 7-diazaspiro[3.4]octan-5-yl)-2-fluoro-N-methylbenzamide

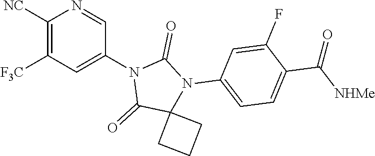 [SMILES: CNC(=O)c1ccc(N2C(=O)N(c3cnc(C#N)c(C(F)(F)F)c3)C(=O)C23CCC3)cc1F]

4-(7-(6-cyano-5-(trifluoromethyl)pyridin-3-yl)-6,8-dioxo-5,7-diazaspiro[3.4]octan-5-yl)-2-fluoro-N-methylbenzamide

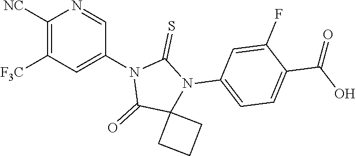 [SMILES: N#Cc1ncc(N2C(=O)C3(CCC3)N(c3ccc(C(=O)O)c(F)c3)C2=S)cc1C(F)(F)F]

4-(7-(6-cyano-5-(trifluoromethyl)pyridin-3-yl)-8-oxo-6-thioxo-5,7-diazaspiro[3.4]octan-5-yl)-2-fluorobenzoic acid

The possible impurities mentioned above are found to be less than 0.15% in the apalutamide produced according to the process of the present application.

Apalutamide and its impurities can be analyzed using high performance liquid chromatography (HPLC), such as with a liquid chromatograph equipped with variable wavelength UV-detector and the method described below:

| Column | YMC Triart C18 150 mm × 4.6 mm, 3.0 μm | | |
|---|---|---|---|
| Flow | 0.9 mL/min | | |
| Load | 10 μL | | |
| Column oven temp | 45° C. | | |
| Detection | 273 nm | | |
| Diluent | Acetonitrile:Water 800:200 v/v | | |
| Elution | Gradient | | |
| Sample concen- | 0.5 mg/mL | | |
| tration | | | |
| Run time | 70 min | | |
| Sample rack | 10° C. | | |
| temperature | | | |
| Needle wash | Diluent | | |
| solution | | | |
| | Preparation of Mobile phase-A: | | |
| | Water (900 mL), Acetonitrile (100 mL) and ortho- | | |
| | phosphoric acid (250 μL) were mixed and filtered | | |
| | through 0.45μ membrane filter. | | |
| | Preparation of Mobile phase-B: | | |
| | Acetonitrile (900 mL) and water (100 mL) were | | |
| | mixed and filtered through 0.45μ membrane filter. | | |
| Gradient: | Time (min) | % M.P-A | % M.P-B |
| | 0 | 80 | 20 |
| | 5 | 80 | 20 |
| | 40 | 40 | 60 |
| | 50 | 10 | 90 |
| | 60 | 10 | 90 |
| | 62 | 80 | 20 |
| | 70 | 80 | 20 |

### EXAMPLES

### Example-1: Preparation of 4-((1-cyanocyclobutyl)amino)-2-fluoro-N-methylbenzamide

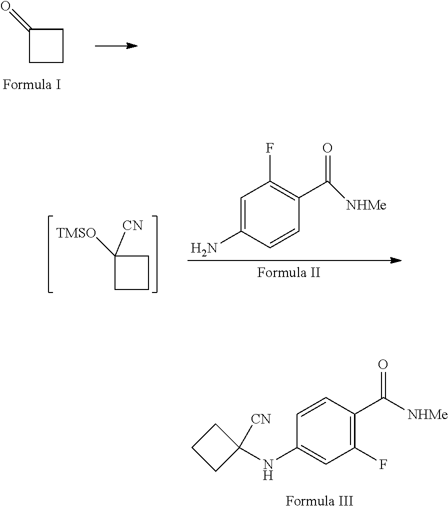

4-amino-2-fluoro-N-methylbenzamide (20 g) and acetonitrile (100 mL) were charged into a round bottom flask at 27° C. The reaction mass was stirred for 5 minutes. Cyclobutanone (9.59 g) and zinc chloride (8.1 g) were added to the reaction mass at 27° C. The reaction mass was cooled to 2° C. Trimethylsilane carbonitrile (TMSCN) (20.6 g) was added to the reaction at 2° C. The reaction mass was stirred for 8 hrs at 5° C. The reaction mass was stirred for 3hrs at 26° C. Water (200 mL) was added to the reaction mass and stirred for 1 hr. The reaction mass was filtered and washed with water (40 mL). The reaction mass was suck dried for 10 minutes. Water (200 mL) was added to the reaction mass and stirred for 4 hrs. The reaction mass was filtered and washed with water (40 mL). The solid was dried under vacuum at 58° C. Product weight: 24.2 g; Yield: 82.31%; Purity by HPLC: 99.39%

### Example-2: Preparation of Apalutamide

4-((1-cyanocyclobutyl)amino)-2-fluoro-N-methylbenzamide (5 g), toluene (50 mL), 5-isothiocyanato-3-(trifluoromethyl)picolinonitrile (9.27 g), N,N-dimethyl acetamide (10 mL), triethylsilylchloride (9.14 g) were charged into a round bottom flask at 25° C. The reaction mass was heated to 60° C. The reaction mass was stirred for 23 hrs at 65° C. 2M HCl (15 mL) was added to the reaction mass at 25° C. The reaction mass was heated to 53° C. The reaction mass was stirred for 6hrs at 58° C. The reaction mass was evaporated under vacuum at 58° C. Isopropyl alcohol (50 mL) and apalutamide seed material (0.05 g) were added to the reaction mass at 50° C. The reaction mass was stirred for 20 minutes at 50° C. Water (35 mL) was added to the reaction mass and stirred for 9 hrs at 28° C. The reaction mass was filtered under vacuum and washed with Isopropyl alcohol (50 mL). The reaction mass was suck dried for 30 minutes. Water (50 mL) was added to the reaction mass and stirred for 3 hrs. The reaction mass was filtered and washed with water (15 mL).The solid was dried under vacuum at 65° C. The obtained apalutamide and isopropyl alcohol (175 mL) were charged into a round bottom flask at 25° C. The reaction mass was heated to 72° C. and stirred for 1 hr. The reaction mass was filtered to make it clear and free of unwanted particles. The reaction mass was stirred for 12 hrs at 28° C. The reaction mass was filtered under vacuum and washed with Isopropyl alcohol (25 mL). The reaction mass was suck dried for 30 minutes. The solid was dried under vacuum at 65° C. Product weight: 7.1 g; Yield: 73.58%.

### Example-3: Preparation for the Purification of Apalutamide

Apalutamide (2 g) and isopropyl alcohol (40 mL) were charged into a round bottom flask at 25° C. The reaction mass was heated to 75° C. and stirred for 30 minutes. The reaction mass was stirred for 4 hrs at 28° C. The reaction mass was filtered under vacuum and washed with Isopropyl alcohol (4 mL). The solid was dried under vacuum at 65° C. Product weight: 1.8 g; Yield: 90%. Purity by HPLC: 99.8%

### Example-4: Preparation of Amorphous Form of Apalutamide

Apalutamide (30 g) was dissolved in methanol (500 mL) at 52° C. The resulted solution was filtered under vacuum to make particle free. The clear solution was subjected to spray drying under nitrogen at a feed rate of 5 g/min and feed solution temperature was 30° C. Nitrogen was used as atomizing gas. Nitrogen inlet temperature was kept at 85° C. and the outlet temperature was kept at 45° C. Thus obtained product was further dried under VTD at 30° C. for 16 hours to obtain the title compound. Yield: 69.6%.

### Example-5: Preparation of Crystalline Apalutamide

Crystalline Apalutamide (250 g) was dissolved in n-Butanol (2500 ml) at 92° C. The resulting solution was seeded with crystalline Apalutamide (2.5 g). The reaction mixture was stirred for 6 hours at 28° C. The reaction mixture was cooled to 4° C. and stirred for 3 hours.

The reaction mixture was filtered and washed with n-butanol (500 mL). The solid was dried under vacuum at 65° C. Purity by HPLC: 99.92%; Yield: 90%

### Example-6: Preparation of Crystalline Apalutamide

n-Butanol (25 ml) was added to crystalline Apalutamide (5 g) at 28° C. The resulting mixture was stirred for 16 hours at 28° C. The reaction mixture was filtered and washed with n-butanol (10 mL). The solid was dried under vacuum at 60° C. Purity by HPLC: 99.86%; Yield: 92%

### Example-7: Preparation of Crystalline Apalutamide

Apalutamide (5 g) was dissolved in methanol (21.25 ml) at 53° C. Water (50 mL) was added to the resulting solution. The reaction mixture was stirred for 24 hours at 53° C. The reaction mixture was cooled to 26° C. The reaction mixture was filtered and washed with water (12.50 mL). The solid was dried under vacuum at 65° C. Yield:70%

### Example-8: Preparation of Crystalline Apalutamide

Crystalline Apalutamide (5 g) was dissolved in diisopropyl ether (50 ml) at 58° C. The resulting reaction mixture was cooled to 28° C. The reaction mixture was filtered. The solid was dried under vacuum at 60° C.

### Example-9: Preparation of Crystalline Apalutamide

Apalutamide (5 g) was dissolved in isobutyl acetate (25 ml) at 92° C. The reaction mixture was cooled to 28° C. and stirred for 2 hours. The reaction mixture was filtered and washed with isobutyl acetate (5 ml). The solid was dried under vacuum at 65° C. Purity by HPLC: 99.90%; Yield: 70%

### Example-10: Preparation of Crystalline Apalutamide

Crystalline Apalutamide (5 g) was dissolved in n-Butanol (50 ml) at 91° C. The reaction mixture was cooled to 46° C. and stirred for 1 hour. n-Heptane (50 mL)was added to the reaction mass. The reaction mixture was stirred for 18 hours at 31° C. The reaction mixture was filtered and washed with n-Heptane (5 mL). The solid was dried under vacuum at 65° C. Purity by HPLC: 99.88%; Yield: 91

### Example-11: Preparation of Crystalline Apalutamide

Crystalline Apalutamide (5g) was dissolved in n-pentanol (25 ml) at 90° C. The reaction mixture was cooled to 28° C. The reaction mixture was filtered and washed with n-pentanol (10 mL). The solid was dried under vacuum at 65° C. Purity by HPLC: 99.9%; Yield: 80%

### Example-: 12 Preparation of Crystalline Apalutamide

Crystalline Apalutamide (5 g) was dissolved in methyl tert-butyl ether (MTBE) (25 ml) at 54° C. The reaction mixture was cooled to 28° C. The reaction mixture was filtered and washed with MTBE (5 mL). The solid was dried under vacuum at 65° C. Purity by HPLC: 99.83%; Yield: 92%

### Example-13: Preparation of Crystalline Apalutamide

Crystalline Apalutamide (5 g) was dissolved in isobutyl acetate (25 ml) at 82° C. The reaction mixture was cooled to 50° C. n-heptane (50 mL) was added to the reaction mass at 50° C. The reaction mixture was cooled to 28° C. The reaction mixture was filtered and washed with n-heptane (5 mL). The solid was dried under vacuum at 65° C. Purity by HPLC: 99.86%; Yield: 91

We claim:

1. A process for the preparation of apalutamide, the process comprising reacting formula III with formula IV in the presence of neutralizing agent followed by treating with acid to obtain Apalutamide.

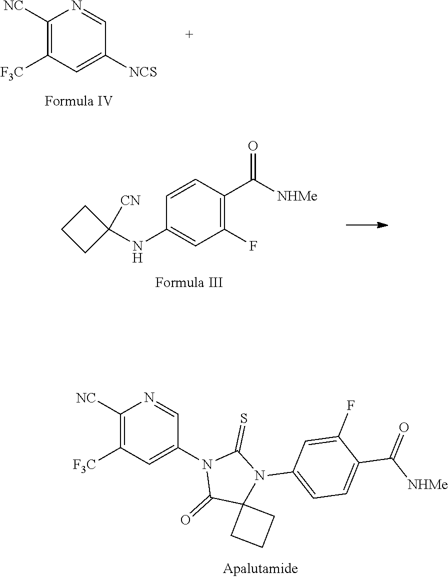

2. The process of claim 1, wherein neutralizing agent selected from triethylsilylchloride, trimethylsilyl chloride, zinc chloride, aluminium chloride, iron chloride, sodium chloride, acetic acid, ammonium chloride or mixture thereof.

3. The process of claim 1, wherein acid selected from hydrochloric acid (HCl), hydrofluoric acid (HF), hydrobromic acid (HBr), hydroiodic acid (HI), sulfuric acid (H2SO4), nitric acid (HNO3), phosphoric acid (H3PO4) or mixture thereof.

4. A process for the preparation of crystalline form of apalutamide characterized by a PXRD pattern comprising peaks at about 12.1°, 16.0°, 16.7°, 20.1°20.3°±0.1°2θ, comprising the steps of:
a) providing apalutamide in solvent selected from n-butanol, methanol, diisopropyl ether, isobutyl acetate, n-pentanol or mixture thereof; and
b) isolating crystalline form of apalutamide.
